(19) United States
(12) Patent Application Publication
(10) Pub. No.: US 2026/0115477 A1
(43) Pub. Date: Apr. 30, 2026

(54) SYSTEMS AND METHODS FOR DELIVERING AND VISUALIZING EFFECTS OF ELECTRICAL STIMULATION

(71) Applicant: Boston Scientific Neuromodulation Corporation, Valencia, CA (US)

(72) Inventors: Adarsh Jayakumar, Valencia, CA (US); Nathan Maas, Simi Valley, CA (US); Chirag Shah, Valencia, CA (US); Sarvani Grandhe, Valencia, CA (US)

(21) Appl. No.: 19/371,605

(22) Filed: Oct. 28, 2025

Related U.S. Application Data
(60) Provisional application No. 63/713,506, filed on Oct. 29, 2024.

(51) Int. Cl. A61N 1/36; A61N 1/05

(52) U.S. Cl. CPC A61N 1/36139; A61N 1/0556

(57) ABSTRACT

A cable for an electrical stimulation system includes a connector body defining a connector lumen; connector contacts disposed or disposable along the connector lumen for electrically coupling to terminals of an electrical stimulation lead or lead extension wherein the connector contacts include first connector contacts and at least one second connector contact; a stimulator cord for coupling to a stimulator and including stimulator contacts disposed along an end portion, wherein the stimulation contacts are electrically coupled to the first connector contacts, but not to any of the at least one second connector contact; and one or more sensor cords n for coupling to an amplifier or signal acquisition unit, an end portion of each of the one or more sensor cords including a connector electrically coupled to at least one of the at least one second connector contact when the sensor cord is coupled to the connector body.

### CROSS-REFERENCE TO RELATED APPLICATIONS

This application claims the benefit under 35 U.S.C. § 119(e) of U.S. Provisional Patent Application Ser. No. 63/713,506, filed Oct. 29, 2024, which is incorporated herein by reference.

### FIELD

The present disclosure is directed to the area of electrical stimulation systems and methods of making and using the systems. The present disclosure is also directed to visualizing the effects of electrical stimulation, including sensing using electrodes.

### BACKGROUND

Implantable electrical stimulation systems have proven therapeutic in a variety of diseases and disorders. For example, peripheral nerve stimulation has been used to treat chronic pain syndrome, incontinence, and a number of other disorders, diseases, and symptoms.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a cable for an electrical stimulation system. The cable includes a connector body defining a connector lumen configured for receiving a proximal end of an implantable electrical stimulation lead or of a lead extension coupled or coupleable to the implantable electrical stimulation lead; a plurality of connector contacts disposed or disposable along the connector lumen and configured for electrically coupling to terminals of the electrical stimulation lead or lead extension when the proximal end of the electrical stimulation lead or lead extension is received in the connector lumen, wherein the plurality of connector contacts includes a plurality of first connector contacts and at least one second connector contact, wherein each of the at least one second connector contact is not one of the first connector contacts; a stimulator cord having a first end portion coupled or coupleable to the connector body and a second end portion configured for coupling to a stimulator, the stimulator cord including a plurality of stimulator contacts disposed along the second end portion, wherein the stimulation contacts are electrically coupled to the first connector contacts, but not to any of the at least one second connector contact, when the stimulator cord is coupled to the connector body; and one or more sensor cords, each of the one or more sensor cords having a first end portion coupled or coupleable to the connector body and a second end portion configured for coupling to an amplifier or signal acquisition unit, the second end portion of each of the one or more sensor cords including a connector electrically coupled to at least one of the at least one second connector contact when the sensor cord is coupled to the connector body.

In at least some aspects, the connector body includes a plurality of the connector lumens. In at least some aspects, the cable includes a plurality of the stimulator cords. In at least some aspects, the cable includes a plurality of the sensor cords. In at least some aspects, each of the at least one second connector contact is electrically isolated from the first connector contacts. In at least some aspects, at least one of the at least one second connector contact is user-selectable from the connector contacts.

Another aspect is an electrical stimulation system, including any of the cables described above; the implantable electrical stimulation lead configured to electrically couple to the cable and including a plurality of electrodes; and the stimulator configured to electrically couple to the stimulator cord of the cable.

In at least some aspects, the implantable stimulation lead includes an implantable cuff lead. In at least some aspects, the stimulator is an external stimulator. In at least some aspects, the electrical stimulation system further includes the amplifier configured to electrically couple to the one or more sensor cords of the cable.

In at least some aspects, the electrical stimulation system further includes the signal acquisition unit configured to electrically couple to the one or more sensor cords of the cable. In at least some aspects, the electrical stimulation system is configured to sense electrical signals from tissue using one or more of the electrodes of the electrical stimulation system and deliver the electrical signals to the signal acquisition unit via the cable and the one or more sensor cords of the cable. In at least some aspects, the electrical stimulation system is configured to analyze the sensed electrical signals. In at least some aspects, the electrical stimulation system is configured to alter one or more stimulation parameters in response to the analysis of the sensed electrical signals. In at least some aspects, the at least one second connector contact is electrically isolated from the stimulator when the stimulator is electrically coupled to the cable.

A further aspect is a method for stimulating tissue of a patient using any of the electrical stimulation systems described above. The method includes electrically coupling the electrical stimulation lead to the cable; electrically coupling the cable to the stimulator; generating, by the stimulator, stimulation signals; and delivering the stimulation signals to the tissue through the stimulator cord of the cable to one or more of the electrodes of the electrical stimulation lead for stimulation of the tissue adjacent the one or more of the electrodes of the electrical stimulation lead.

In at least some aspects, the method further includes, after the delivering, sensing electrical signals from the tissue using one or more of the electrodes of the electrical stimulation lead and delivering the electrical signals through the at least one sensor cord of the cable to an amplifier. In at least some aspects, the method further includes, after the delivering, sensing electrical signals from the tissue using one or more of the electrodes of the electrical stimulation lead and delivering the electrical signals through the at least one sensor cord of the cable to a signal acquisition unit. In at least some aspects, the method further includes analyzing the delivered electrical signals. In at least some aspects, the method further includes altering one or more stimulation parameters in response to the analysis of the delivered electrical signals.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of an embodiment of an electrical stimulation system;

FIG. 2 is a schematic view of one embodiment of a cuff lead for electrical stimulation;

FIG. 3 is a schematic view of a distal portion of one embodiment of a lead extension configured and arranged to electrically couple to an electrical stimulation lead;

FIG. 4 is a schematic perspective view of one embodiment of a cuff with two sets of sixteen longitudinal electrodes each and two radial electrodes;

FIG. 5 is a schematic view of one embodiment of a cuff, flattened for illustrative purposes, with two sets of eight longitudinal electrodes each and two radial electrodes;

FIG. 6 is a block diagram of another embodiment of an electrical stimulation system;

FIG. 7 is schematic top view of one embodiment of a cable for the electrical stimulation system of FIG. 6;

FIG. 8 illustrates a portion of the cable of FIG. 7 with a switch that alters selection of conductive contacts for coupling to sensor cords;

FIG. 9 illustrates a macroburst using graphs of amplitude versus time for eight electrodes;

FIG. 10 is a cross-sectional view of a cuff disposed around a portion of a nerve illustrating an estimated stimulation region; and

FIG. 11 is a schematic overview of one embodiment of components of an electrical stimulation arrangement according to an embodiment of the present invention.

### DETAILED DESCRIPTION

The present disclosure is directed to the area of electrical stimulation systems and methods of making and using the systems. The present disclosure is also directed to visualizing the effects of electrical stimulation, including sensing using electrodes.

Non-limiting examples of electrical stimulation systems, which can be modified as described herein to include sensing capabilities or modified cables, are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, as well as the other references cited herein, all of which are incorporated by reference in their entireties. Examples of devices for measuring or otherwise sensing an evoked potential (e.g., a neural response) or other electrical signals are found in, for example, U.S. Pat. Nos. 7,385,443; 11,040,202; 11,633,138; U.S. Patent Application Publication Nos. 2022/0007808, 2022/0007980, 2022/0300434; 2023/0173273, 2024/0058611; and U.S. Provisional Patent Application Publication No. 63/655,935, all of which are incorporated herein by reference.

Electrical stimulation systems typically include at least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead.

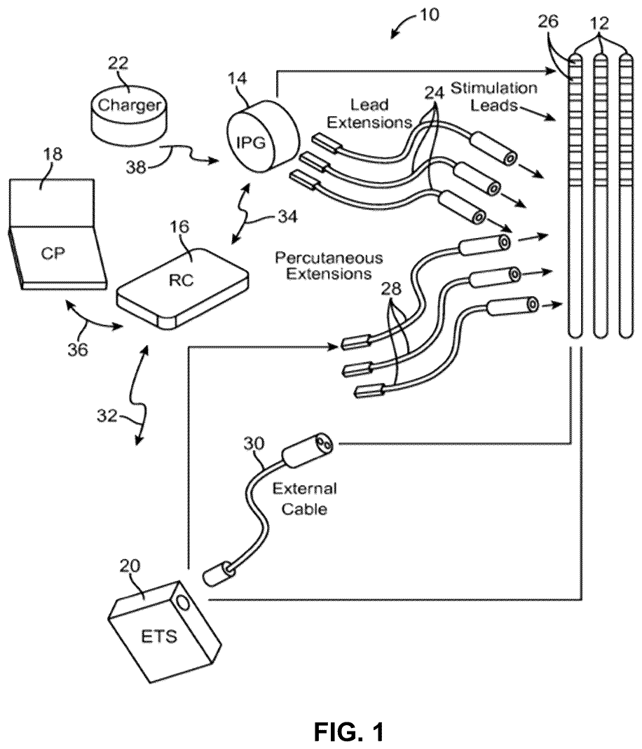

Turning to FIG. 1, one embodiment of an electrical stimulation system 10 includes one or more stimulation leads 12 and an implantable pulse generator (IPG) 14. The stimulation system 10 can also include one or more of an external remote control (RC) 16, a clinician's programmer (CP) 18, an external trial stimulator (ETS) 20, or an external charger 22. The IPG and ETS are examples of stimulators or control modules for the electrical stimulation system. The ETS 20 is a type of external stimulator and will be used herein as an example, but it will be understood that any other external stimulator can be used in the place of the ETS 20.

The IPG 14 or ETS 20 is physically connected, in at least some embodiments, via one or more lead extensions 24 or percutaneous extensions 28, to the stimulation lead(s) 12. In at least some embodiments, each lead carries multiple electrodes 26 arranged in an array. In at least some embodiments, the IPG 14 or ETS 20 includes pulse generation circuitry that delivers electrical stimulation energy in the form of, for example, a pulsed electrical waveform (i.e., a temporal series of electrical pulses) to the electrode array 26 in accordance with a set of stimulation parameters. Examples of stimulation parameters include, but are not limited to, electrode selection, amplitude, pulse width, pulse shape, pulse frequency, pulse arrangement (e.g., pulse bursts, number of pulses per burst, or burst frequency) or the like or any combination thereof. The IPG 14 can be implanted into a patient's body, for example, below the patient's clavicle area or within the patient's buttocks or abdominal cavity or at any other suitable site.

The IPG 14 or ETS 20 can have multiple stimulation channels which may be independently programmable to control the magnitude of the current stimulus from each channel. In at least some embodiments, the IPG 14 or ETS 20 can have any suitable number of stimulation channels including, but not limited to, 2, 3, 4, 6, 8, 12, 16, 32, or more stimulation channels. In various embodiments, the IPG 14 or ETS 20 can have one, two, three, four, or more connector ports, for receiving the terminals of the leads and/or lead/percutaneous extensions 24, 28.

The ETS 20 can be physically connected via an external cable 30 and, in at least some embodiments, the percutaneous lead extensions 28, to the stimulation leads 12. The ETS 20, which can have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is a non-implantable device. In at least some embodiments, the ETS 20 is used on a trial basis after the neurostimulation leads 12 have been implanted and prior to implantation of the IPG 14, to test the responsiveness of the stimulation that is to be provided. In at least some embodiments, the ETS 20 can be used for determining stimulation parameters or initial stimulation parameters, which can be modified using the IPG 14. Any functions described herein with respect to the IPG 14 can likewise be performed with respect to the ETS 20 in at least some embodiments.

The RC 16 can be used to telemetrically communicate with or control the IPG 14 or ETS 20 via a uni- or bi-directional wireless communications link 32. The RC 16 or CP 18 can be used to telemetrically communicate with or control the IPG 14 or ETS 20 via a uni- or bi-directional communications link 34. In at least some embodiments, such communication or control allows the IPG 14 or ETS 20 to be turned on or off and to be programmed with different stimulation parameter sets.

The IPG 14 or ETS 20 may also be operated to modify the programmed stimulation parameters to actively control the characteristics of the electrical stimulation energy output by the IPG 14 or ETS 20. In at least some embodiments, the CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14 or ETS 20.

In at least some embodiments, the CP 18 may perform this function by indirectly communicating with the IPG 14 or ETS 20, through the RC 16, via a wireless communications link 36. Alternatively, in at least some embodiments, the CP 18 may directly communicate with the IPG 14 or ETS 20 via a wireless communications link (not shown). In at least some embodiments, the stimulation parameters provided by the CP 18 are also used to program the RC 16, so that the stimulation parameters can be subsequently modified by operation of the RC 16 in a stand-alone mode (i.e., without the assistance of the CP 18).

Stimulation provided by IPG 14 or ETS 20 is typically provided by pulses, each of which may include a number of phases. Stimulation parameters typically include amplitude (current I, although a voltage amplitude V can also be used); frequency (F); pulse width (PW); the electrodes 26 selected to provide the stimulation; the polarity of such selected electrodes, i.e., whether they act as anodes that source current to the tissue or cathodes that sink current from the tissue; or the like or any combination thereof. In at least some embodiments, these and possibly other stimulation parameters taken together comprise a stimulation program that the stimulation circuitry 229 in the IPG 14 or ETS 20 can execute to provide therapeutic stimulation to a patient.

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited herein, all of which are incorporated herein by reference.

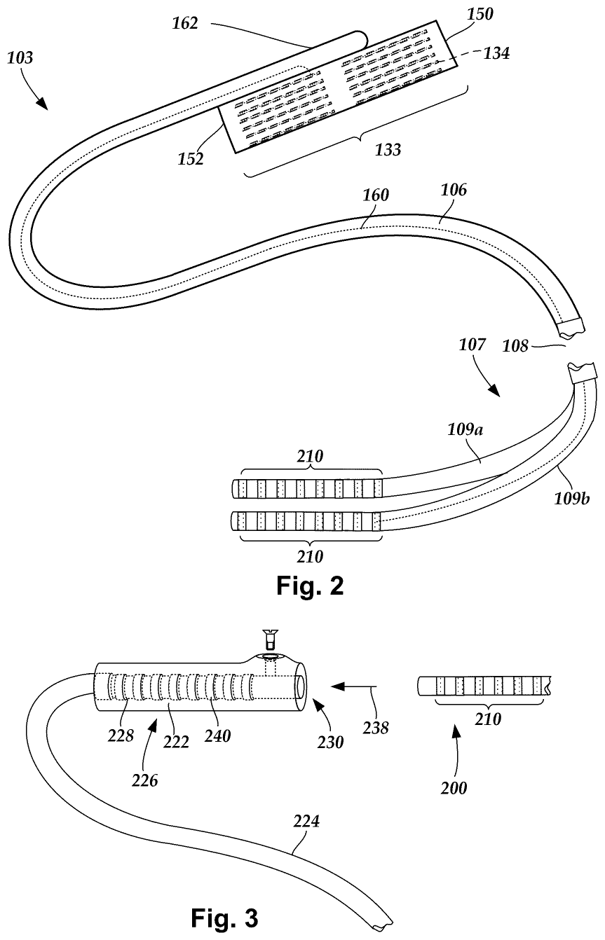

FIG. 2 illustrates schematically one embodiment of a cuff lead 103 coupleable to the control module, such as the IPG 14 or ETS 20 of FIG. 1. The lead 103 includes a mount 162 and a cuff 150 with a cuff body 152 and an array of electrodes 133, such as electrode 134. The lead 103 also includes one or more lead bodies 106, coupled to or containing the mount 162, and an array of terminals 210 attached to the one or more lead bodies 106. In at least some embodiments, the lead is isodiametric along at least a portion of the longitudinal length of the lead body 106.

In FIG. 2, the lead 103 is shown having a splitter 107 configured and arranged for facilitating coupling of the lead 103 to the control module 102. The splitter 107 includes a splitter connector 108 configured to couple to a proximal end of the lead 103, and one or more proximal tails 109a and 109b configured and arranged to couple to the control module (or another splitter, a lead extension, an adaptor, or the like). The splitter 107 and splitter connector 108 may be part of the lead 103 or may be a separate component that attaches to the lead.

The lead body 106 can be made of, for example, a non-conductive, biocompatible material such as, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end of the lead body 106 to the proximal end of the lead body 106.

Any suitable arrangement of electrodes 134 on the cuff 150 can be used. Examples of cuff leads 103 and electrode arrangements for cuff leads can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0370793; and 2022/0395690; U.S. patent application Ser. No. 18/886,646 and U.S. Provisional Patent Applications Ser. Nos. 63/549,797; 63/668,928; 63/672,566; and 63/691,918, all of which are incorporated herein by reference in their entireties.

Terminals 210 are typically disposed along the proximal end of the lead body 106 of the electrical stimulation system 100 (as well as any splitters, lead extensions, adaptors, or the like) for electrical connection to corresponding connector contacts 240 (FIG. 3) of a lead/peripheral extension 24, 28, an external cable 30, or a control module, such as IPG 14 or ETS 20. The connector contacts 240 are disposed in connectors 222 (FIG. 3) which, in turn, are disposed on the lead/peripheral extension 24, 28, an external cable 30, or a control module, such as IPG 14 or ETS 20.

Electrically conductive wires 160, cables, or the like (only one of which is shown in FIG. 2) extend from the terminals to the electrodes 134. Typically, one or more electrodes 134 are electrically coupled to each terminal. In at least some embodiments, each terminal is only connected to one electrode 134. The electrically conductive wires (“conductors”) 160 (only one of which is illustrated in FIG. 2 for clarity) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

FIG. 2 also illustrates a mount 162, part of the lead body 106, coupled to the cuff 150. The conductors 160 (only one of which is illustrated in FIG. 2 for clarity) from within the lead body 106 are received in the mount 162, which in turn is attached to the cuff 150 such that each conductor passes through the mount 162 for a direct electrical connection with one of the electrodes 134 (e.g., one conductor is electrically connected with one electrode and so on). The mount 162 may be attached using a variety of means such as, but not limited to, molding or adhering the mount 162 to the cuff 150. In other embodiments, the conductors 160 from within the lead body 106 are electrically coupled to the electrodes 134 using jumper, intermediate or transition wires from the lead body 106 to the electrodes 134.

The mount 162 can be offset from the cuff 150, as illustrated in FIG. 2, or in-line with the cuff or in any other suitable arrangement. Non-limiting examples of cuff leads 103 can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0226641; and 2022/0370793;

U.S. patent application Ser. No. 18/886,646 and U.S. Provisional Patent Applications Ser. Nos. 63/549,797; 63/668,928; 63/672,566; and 63/691,918, all of which are incorporated herein by reference in their entireties.

FIG. 3 is a schematic side view of a distal portion of a lead extension 224 that is configured and arranged to couple one or more elongated devices 200 (e.g., the lead body 106, an adaptor, another lead extension, or the like or combinations thereof) to the control module 102. The lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 3, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (e.g., electrodes 134 of FIG. 2) disposed along the lead (e.g., lead 103 of FIG. 2).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end of the lead extension 224 that is opposite to the distal end 226. In at least some embodiments, the conductive wires disposed in the lead extension 224 can be electrically coupled to a plurality of terminals (not shown) disposed along the proximal end of the lead extension 224. In at least some embodiments, the proximal end of the lead extension 224 is configured and arranged for insertion into a connector disposed in another lead extension (or another intermediate device).

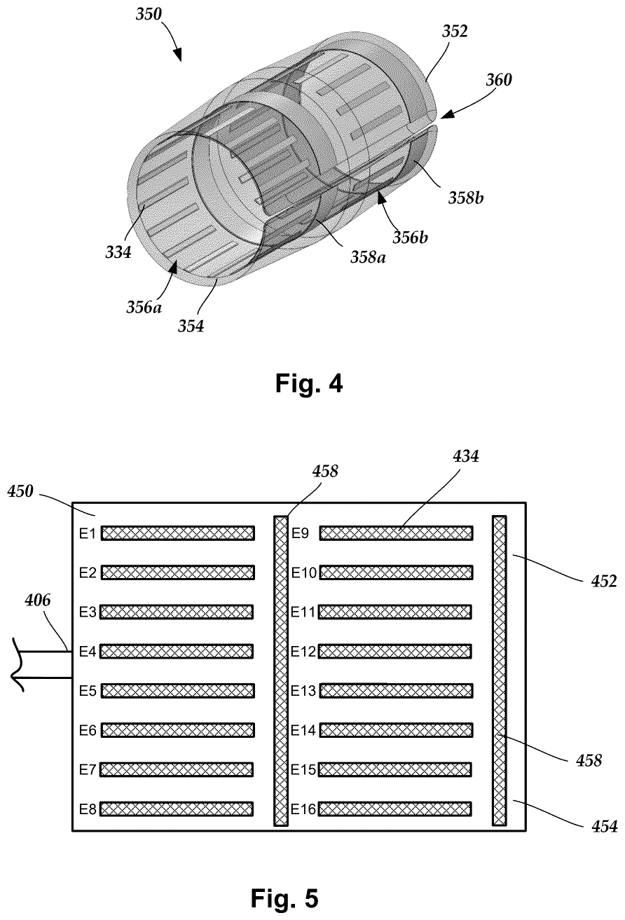

FIG. 4 illustrates one embodiment of a cuff 350 of a cuff lead 103 (FIG. 2). The cuff 350 includes a cuff body 352 with longitudinal electrodes 334 disposed on an interior surface 354 of the cuff body and arranged around the circumference of the cuff body in two sets 356a, 356b. In the illustrated embodiment, each set 356a, 356b includes sixteen longitudinal electrodes 334. Any other suitable number of electrodes can be used including, but not limited to, 16, 20, 25, 28, 32, 36, 40, 48, 50, 64, 80, 100, 120, 128, 150, 200, 250, 256, or more longitudinal electrodes. A cuff lead can have one, two, three, four, or more sets of longitudinal electrodes 334. The number of longitudinal electrodes 334 in a set can be the same for each set or can differ. In the illustrated embodiment, the longitudinal electrodes 334 of each set are aligned longitudinally with electrodes of the other set. In other embodiments, the longitudinal electrodes 334 of each set can be staggered or unaligned with the electrodes of another set.

In addition, the cuff 350 includes two radial electrodes 358a, 358b that wrap around at least 75%, 80%, 90%, or 95% of the circumference of the cuff body 352. The cuff 350 also defines a slit 360 that extends the longitudinal length of the cuff body 352 so that the nerve can be loaded into the interior 362 of the cuff body by opening the slit to fit the cuff body over the nerve. The slit 360 is opened or initially sized to allow the target nerve (not shown) to be slipped, inserted, fed, or otherwise received into the cuff 350 such that the cuff 350 wraps around the target nerve. In at least some embodiments, the slit 360 allows the cuff 350 to be easily moved over and around the target nerve or relative to the target nerve whether rotationally or transitionally.

The electrodes 134, 334, 358a, 358b can be formed using any conductive, biocompatible material. Examples of suitable materials include metals, alloys, conductive polymers, conductive carbon, and the like, as well as combinations thereof. In at least some embodiments, one or more of the electrodes 134, 334, 358a, 358b are formed from one or more of: platinum, platinum alloys such as platinum iridium, palladium alloys such as palladium rhodium, titanium, titanium alloys, nickel alloys, cobalt alloys, nickel/cobalt alloys, stainless steel, tantalum, conductive carbon, conductive plastics, epoxy or other adhesive filled with metallic powder, Nitinol™, or the like or any combination thereof. The electrodes 134, 334, 358a, 358b can be formed by any suitable process including, but not limited to, machining, molding (for example, powdered metal molding), photolithography, additive techniques, stamping, or the like or any combination thereof.

The cuff body 152, 352 can be formed of any suitable biocompatible and biostable non-conductive material including, but not limited to, polymer materials such as silicone, polyurethane, polyetheretherketone (“PEEK”), epoxy, or the like or any combination thereof. In at least some embodiments, the cuff body 152, 352 can have a circular, oval, or any other suitable cross-sectional shape and, at least in some embodiments, may be sufficiently flexible to alter the cross-sectional shape to accommodate the nerve. In at least some embodiments, the electrodes 134, 334, 358a, 358b can be molded with the cuff body 152, 352 or formed by techniques such as etching or ablation of conductive layers, films, or the like. In at least some embodiments, the cuff body 152, 352 has an inner diameter (which can correspond to the largest diameter of a non-circular cuff body) in a range of 0.5 to 5 mm or in a range of 1 to 3 mm. at least some embodiments, the cuff body 152, 352 has a length of at least 5, 10, or 20 mm.

In at least some embodiments, the cuff body 152, 352 can be formed using any suitable technique including, but not limited to, molding, casting, formed in a sheet and then shaped using adhesive as a binder, formed flat and shaped using heat, formed flat and attached to a cuff-shaped scaffold, pressed or extruded into the cuff shape, or the like or any combination thereof. In at least some embodiments, the electrodes 134, 334, 358a, 358b can be attached to the cuff body 152, 352 using any suitable technique including, but not limited to, attaching with adhesive, molding (for example, insert molding) into the cuff body, using heat to adhere the electrodes to the cuff body, heating and pressing the electrodes into the cuff body, depositing electrode material on the cuff body and using photolithography and etching, or the like or any combination thereof.

In at least some embodiments, once the cuff 350 has been placed in a desired position relative to the target nerve, the edges of the cuff body 352 defining the slit 360 can be sutured to capture the target nerve without undesirably compressing the target nerve. In at least some embodiments, suture holes (not shown) are optionally incorporated into the edges of the cuff 350 to allow for closing or partially closing the cuff 350 around the target nerve. Any other suitable arrangement, method, or technique can be used to secure the cuff 350 to the target nerve.

FIG. 5 illustrates another embodiment of a cuff 450 and distal end of a lead body 406 of a cuff lead 103 (FIG. 2). For illustrative purposes, the cuff 450 is illustrated unwrapped and laid flat. The cuff 450 includes a cuff body 452 with longitudinal electrodes 434 (labeled E1 to E16) disposed on an interior surface 454 of the cuff body and arranged around the circumference of the cuff body in two sets of eight electrodes each (electrodes E1 to E8 and electrodes E9 to E16, respectively). The cuff 450 also includes two radial electrodes 458.

Although not limited to cuff leads, a cuff lead will be used herein for illustration of the methods and systems. A cuff of a cuff lead can be implanted around a peripheral nerve, such as the vagus nerve, to stimulate (e.g., by delivering electrical pulses) the nerve. In at least some embodiments, the stimulation can generate actions potentials or block transmission of action potentials to either the brain or to one or more peripheral organs or other nerve terminals. In at least some embodiments, stimulation can be used to modulate the function of the brain, the peripheral organ(s), or both. As an example, electrical stimulation or neuromodulation of the vagus nerve (or any other suitable nerve) can be used to alleviate various disease or disorder conditions of the brain, the peripheral organ(s), or other portions of the body.

It can be useful to visualize electrical stimulation being delivered to tissue. For example, it can be useful to visualize electrical stimulation during programming of the electrical stimulation system, particularly, as the stimulation parameters are altered during the programming and testing process or procedure. It can be useful to visualize electrical stimulation in real time. In at least some embodiments, an electrical stimulation system can show in real time where and when stimulation is happening for each selection of stimulation parameters.

In at least some embodiments, visualization of electrical stimulation can be facilitated by sensing neural response to the electrical stimulation by, for example, monitoring or measuring electrical signals from neural tissue, nerve, spinal cord, brain tissue, or other tissue that is stimulated or otherwise responsive to the electrical stimulation. In at least some embodiments, these neural signals can be relatively weak, for example, less than 1 mV in amplitude. As described herein, electrodes of an electrical stimulation lead, such as a lead 12 of FIG. 1 or a cuff lead 103 of FIG. 2, can be used to sense the neural signals. In at least some embodiments, as further described herein, the electrodes used for sensing can be electrically separated or isolated from the stimulator, such as IPG 14 or ETS 20 of FIG. 1, or from the electrodes used to deliver stimulation or from the signals generated by the stimulator and delivered to the stimulation electrodes. In at least some embodiments, such separation or isolation can reduce or avoid cross-talk with, or saturation by, signals generated by the stimulator.

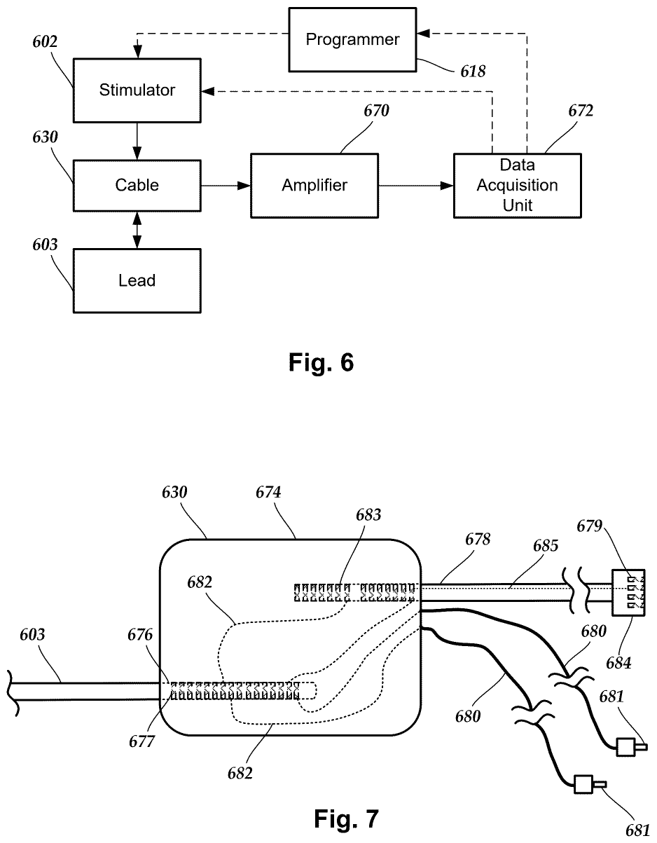

FIG. 6 illustrates one embodiment of an electrical stimulation system 600 that includes a stimulator 602 (such as IPG 14 or ETS 20 of FIG. 1), a cable 630 (such as external cable 30 of FIG. 1), at least one electrical stimulation lead 603 (such as leads 12 of FIG. 1 or cuff lead 103 of FIG. 2), an optional amplifier 670, a data acquisition unit 672, and an optional programmer 618 (such as CP 18 or RC 16 of FIG. 1). In at least some embodiments, one or more electrodes of the at least one electrical stimulation lead 603 can be used to sense electrical signals from patient tissue. It will be recognized that a data acquisition unit 672 may include an amplifier, instead of (or in addition to) the optional amplifier 670. An amplifier can amplify electrical signals (e.g., neural signals) sensed using, for example, one of more electrodes of the electrical stimulation lead(s) 603, one or more other sensors, or any combination thereof.

In at least some embodiments, the data acquisition unit 672 includes a processor and a memory. Any suitable processor and memory can be used. Any suitable data acquisition unit can be used. In at least some embodiments, sensed electrical signals (e.g., sensed neural signals) obtained using the at least one stimulation lead 603 or other sensor can be stored in the memory of the data acquisition unit 672. In at least some embodiments, the data acquisition unit 672 can provide the sensed electrical signals to a programmer 618, such as CP 18 or RC 16 of FIG. 1, or other device for consideration or analysis by a user (e.g., one or more of the patient, a clinician, a caregiver, or the like).

In at least some embodiments, the data acquisition unit 672 (or the programmer 618 or other device) is configured for analyzing the sensed electrical signals. In at least some embodiments, the data acquisition unit 672 (or the programmer 618 or other device) can determine or identify one or more features of the sensed electrical signals, such as, for example, an area under a curve of the sensed electrical signal; a range of an amplitude of the curve in a defined time window; a length of the curve of the sensed electrical signal; an energy content of the sensed electrical signal; a range of a derivative of the sensed electrical signal in a predefined time window; a time delay of a specified signal peak of the sensed electrical signal; power of the sensed electrical signal in a predefined frequency band; variation of a frequency peak with highest energy in the sensed electrical signal in a predefined frequency band; coherence changes in the sensed electrical signal; a quality factor for a specified signal peak of the sensed electrical signal in a predefined time window; full width at half amplitude (FWHM) for a specified signal peak of the sensed electrical signal in a predefined time window; entropy of the sensed electrical signal; or spectral entropy of the sensed electrical signal. In at least some embodiments, the data acquisition unit 672 can provide the features or other analysis to the programmer 618 or other device for consideration or analysis by the programmer 618 or other device or by a user (e.g., one or more of the patient, a clinician, a caregiver, or the like) who may alter stimulation based on the sensed electrical signals or features or other analysis.

In at least some embodiments, the data acquisition unit 672 is configured for analyzing the sensed electrical signals and identifying, proposing, selecting, or determining one or more changes to the one or more stimulation parameters, when appropriate, needed, desired, or useful. In at least some embodiments, the stimulation parameter change(s) is delivered to the stimulator 602 (directly or through a programmer, such as CP 18 or RC 16 or the like, or other device) for automatic implementation or for implementation after approval by a user (e.g., one or more of the patient, a clinician, a caregiver, or the like). In at least some embodiments, the identified stimulation parameter changes are directed to a programmer, such as CP18 or RC 16, or other device for consideration, approval, selection, or implementation by a user (e.g., one or more of the patient, a clinician, a caregiver, or the like).

Non-limiting examples of methods for analyzing clinical effects and stimulation parameters to identify alterations to stimulation parameters can be found at, for example, U.S. Pat. Nos. 8,326,433; 8,675,945; 8,831,731; 8,849,632; 8.958,615; 9,227,074; 9,248,296; 9,358,398; 9,474,903; 10,071,249; 10,265,528; 10,357,657; 10,369,364; 10,603,498; and 10,716,505; U.S. Patent Application Publications Nos. 2009/0287272; 2009/0287273; 2012/0314924; 2013/0116744; 2014/0122379; 2014/0243926; 2014/0276707; 2014/0277282; 2014/0277284; 2015/0066111; 2018/0264278; 2020/0376263; 2020/0398057; 2021/0023374; 2022/0300434; 2022/0339448; 2023/0181090; 2024/0157151; and 2024/0198110; and U.S. patent application Ser. Nos. 18/645,034 and 18/763,676, all of which are incorporated herein by reference in their entireties.

As an example, the analysis of the sensed electrical signals can include identifying one or more features of the electrical signals. Examples of such features include, but are not limited to, an area under curve of the electrical signal; a range of an amplitude of the curve in a defined time window; a length of the curve of the electrical signal; an energy content of the electrical signal; a range of a derivative of the electrical signal in a predefined time window; a time delay of a specified signal peak of the electrical signal; power of the electrical signal in a predefined frequency band; variation of a frequency peak with highest energy in the electrical signal in a predefined frequency band; coherence changes in the electrical signal; a quality factor for a specified signal peak of the electrical signal in a predefined time window; or full width at half amplitude (FWHM) for a specified signal peak of the electrical signal in a predefined time window; mutual information in the electrical signals; entropy of the electrical signal; spectral entropy of the electrical signal; a standard deviation of any of the previous metrics; or the like or any combination thereof. The features may be evaluated in the frequency domain or time domain.

In at least some embodiments, the electrical stimulation system 600 defines specific first electrodes of the electrical stimulation lead 603 for stimulation and also defines different, specific second electrodes of the electrical stimulation lead 603 for sensing electrical signals from the patient tissue. In at least some embodiments, the cable 630 is arranged so that one or more of the electrodes of the electrical stimulation lead 603 are not directly connected to the stimulator 602. In at least some embodiments, these one or more electrodes (e.g., the second electrodes) are electrically isolated from direct connection to nodes used for stimulation. In at least some embodiments, the cable 630 is used to designate one or more specific second electrodes of the electrical stimulation lead 103 for sensing. In at least some embodiments, the electrical stimulation system 600 prevents the second electrodes of the electrical stimulation lead 103 used for sensing from being used for stimulation. In at least some embodiments, the cable 630 prevents the second electrodes of the electrical stimulation lead 103 used for sensing from being used for stimulation. In at least some embodiments, the electrical stimulation system 600 or the cable 630 can be used to alter or select which of the electrodes are selected for sensing. For example, by use of a switch or other mechanism, the cable 630 can be used to select which of the electrodes are selected for sensing.

FIG. 7 illustrates one embodiment of a cable 630 for coupling at least one electrical stimulation lead 603 (FIG. 6) to a stimulator 602 (FIG. 6). The cable 630 includes a connector body 674, at least one stimulator cord 678 coupled or coupleable to the connector body 674 and configured for coupling to the stimulator 602, and one or more sensor cords 680 coupled or coupleable to the connector body 674 and configured for coupling to the amplifier 670 (FIG. 6) or the signal acquisition unit 672 (FIG. 6) or other device. Non-limiting examples of cables (e.g., operating room (OR) cables or external cables) that can be modified to include one or more sensor cords, as disclosed herein, include, but are not limited to, cables disclosed in U.S. Pat. Nos. 7,548,788; 9,101,775; 9,662,506; 10,130,806; and 10,195,446; and U.S. Patent Application Publication No. 2016/0166836, all of which are incorporated herein by reference in their entireties.

The analysis can identify, for example, efficacy of the electrical stimulation, strength of patient response to stimulation, presence or absence of one or more side effects, patient activity, patient posture, or the like or any combination thereof. In at least some embodiments, the electrical stimulation system can utilize the analysis to identify, propose, select, or determine one or more changes to one or more stimulation parameters to alter the stimulation in a desired manner. For example, the change may have the intended effect of improving efficacy of the stimulation or may alter the stimulation based on patient activity or patient posture or the like or any combination thereof.

The connector body 674 defines one or more connector lumens 676 for receiving a proximal end of an implantable electrical stimulation lead 603 or a lead extension (for example, lead/proximal extensions 24, 28 of FIG. 1 or lead extension 224 of FIG. 3). The connector body 674 includes connector contacts 677 disposed, or disposable, along the connector lumen(s) 676. The connector contacts 677 are arranged for electrically coupling to terminals on the electrical stimulation lead or lead extension when the proximal end of the electrical stimulation lead or lead extension is received in the connector lumen. In at least some embodiments, the connector contacts 677 are arranged to make contact with the terminals on the electrical stimulation lead or lead extension automatically as the end of the electrical stimulation lead or lead extension is inserted into the connector lumen 676. In at least some other embodiments, the connector contacts 677 are moveable to engage with the terminals on the end of the electrical stimulation lead or lead extension. In at least some embodiments, the connector body 674, connector lumens 676, and connector contacts 677 can be the corresponding elements of any of the cables disclosed in the references cited herein.

The cable 630 includes at least one stimulator cord 678 having a first end portion coupled or coupleable to the connector body 674 and a second end portion configured for coupling to the stimulator 602 (FIG. 6). The cable 730 can have one, two, three, four, or more stimulator cords 678. In at least some embodiments, the stimulator cord 678 includes stimulator contacts 679 (only a portion of which are illustrated in FIG. 7) disposed along the second end portion or as part of a connector 684 on the second end portion of the stimulator cord 678. The stimulator contacts 679 are arranged for electrical coupling to the stimulator 602. Alternatively, the stimulator cord(s) 678 can be hardwired to the stimulator 602.

In at least some embodiments, the stimulator cord 678 is hardwired to the connector body 674, as illustrated in FIG. 7. In at least some other embodiments, the stimulator cord 678 is removably engaged with the connector body 674 using, for example, terminals, contacts, and lumens like those used for the coupling the electrical stimulation lead or lead extension to the cable 630, as described herein. The stimulator cord 678 can be the corresponding element of any of the cables disclosed in the references cited herein.

The cable 630 also includes one or more sensor cords 680, each of which has a first end portion coupled or coupleable to the connector body 674 and a second end portion configured for coupling to the amplifier 670 (FIG. 6) or the signal acquisition unit 672 (FIG. 6) or other device. The cable 730 can have one, two, three, four, or more sensor cords 680. In at least some embodiments, a connector 681 with one or more terminals or contacts is disposed on the second end portion of each sensor cord 680 for attachment to the amplifier 670 or the signal acquisition unit 672. In at least some embodiments, the connector 681 is a low noise connector, a safe touch connector, any other suitable connector with one or more terminals/contacts, or the like or any combination thereof. In other embodiments, at least one sensor cord 680 is hardwired to the amplifier 670 or the signal acquisition unit 672 or other device.

In at least some embodiments, the one or more sensor cords 680 are hardwired to the connector body 674, as illustrated in FIG. 7. In at least some other embodiments, the one or more sensor cords 680 are removably engaged with the connector body 674 using, for example, terminals, contacts, and lumens like those used for the coupling the electrical stimulation lead or lead extension to the cable 630, as described herein.

In operation, the cable 630 electrically couples selected electrodes of an electrical stimulation lead 603 to the stimulator 602 via the stimulator cord(s) 678 and electrically couples one or more other electrodes to the amplifier 670 or signal acquisition unit 672 via the sensor cord(s) 680. In at least some embodiments, a first set of the connector contacts 677 of the cable 630 are electrically coupled to the stimulator contacts 679 on the second end of the stimulator cord(s) 678 and a second set of one or more of the connector contacts 677 of the cable 630 are electrically coupled to the connector(s) 681 on the sensor cord(s) 680. None of the connector contacts 677 are in both the first set and the second set.

In at least some embodiments, this arrangement permits the carrying of current non-inductively from the stimulator 602 through the first set of stimulator contacts 679 of the stimulator cord 678 to the connector contacts 677 of the cable 630 and then to a first set of the electrodes of the electrical stimulation lead 603. In at least some embodiments, this arrangement permits the carrying of electrical signals (e.g., sensor signals or neural signals) non-inductively from a second set of one or more electrodes of the electrical stimulation lead 603 through the second set of one or more stimulator contacts 679 to the connector(s) 681 of the sensor cord(s) 680 and then to the amplifier 670 or signal acquisition unit 672.

In at least some embodiments, conductors 682, such as wires, conductive traces, contacts, any other suitable component(s) or mechanism(s) for non-inductively carrying current, or the like or any combination thereof can be used to non-inductively, electrically couple the connector contacts 677 to the stimulator contacts 679 and the connector(s) 681. As a non-limiting example, FIG. 7 illustrates wires 682 (or conductive traces) extending from the second set of one or more connector contacts 677 to, and through, the sensor cord(s) 680 to the connector(s) 681. As another non-limiting example, FIG. 7 illustrates wires 682 (or conductive traces) electrically coupling the first set of connector contacts 677 to cord contacts 683 with additional conductors, such as wires 685, extending from the cord contacts 683 through the stimulator cord(s) 678 to the stimulator contacts 679. It will be recognized that cord contacts 683 can be used with the sensor cord(s) 680. It will also be recognized that cord contacts 683 can be eliminated with wires (or conductive traces) extending from the first set of connector contacts 677 to the stimulation contacts 679 of the stimulator cord(s) 678.

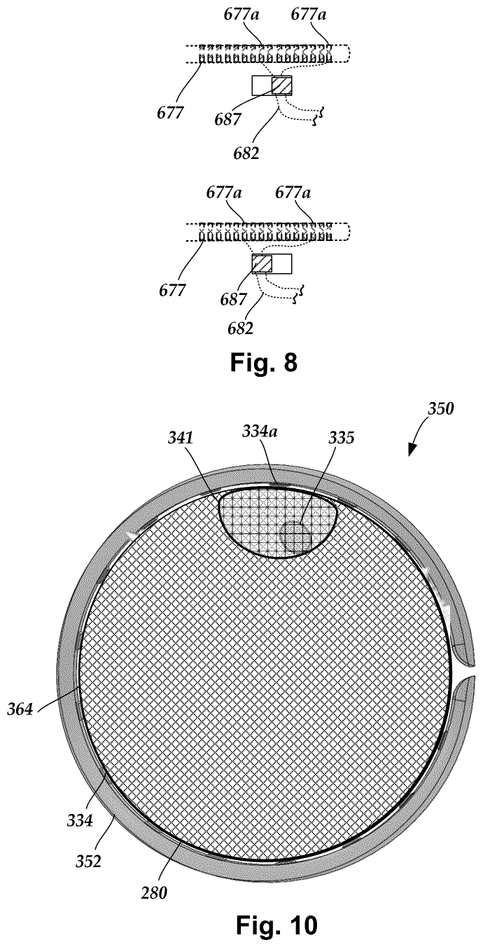

In at least some embodiments, the identity of the connector contacts 677 in the first set of connector contacts electrically coupled to the stimulator contacts 679 and the identity of the connector contact(s) 677 in the second set of one or more connector contacts electrically coupled to the connector(s) 681 of the sensor cord(s) 680 is fixed. In at least some embodiments, in the first set of connector contacts electrically coupled to the stimulator contacts 679 and the identity of the connector contact(s) 677 in the second set of one or more connector contacts electrically coupled to the connector(s) 681 of the sensor cord(s) 680 is user-selectable. For example, in at least some embodiments, as illustrated in FIG. 8, a user can operate a switch 687 or other physical or digital mechanism to select which connector contacts 677 are in the first set and which connector contact(s) 677a are in the second set. FIG. 8 illustrates a switch 687 in two different positions with different selections of connector contacts 677a for the second set (i.e., the connector contacts associated with the sensor cords 680). In at least some of the user-selectable embodiments, there are restrictions on which connector contact(s) 677 are in the second set or in the first set.

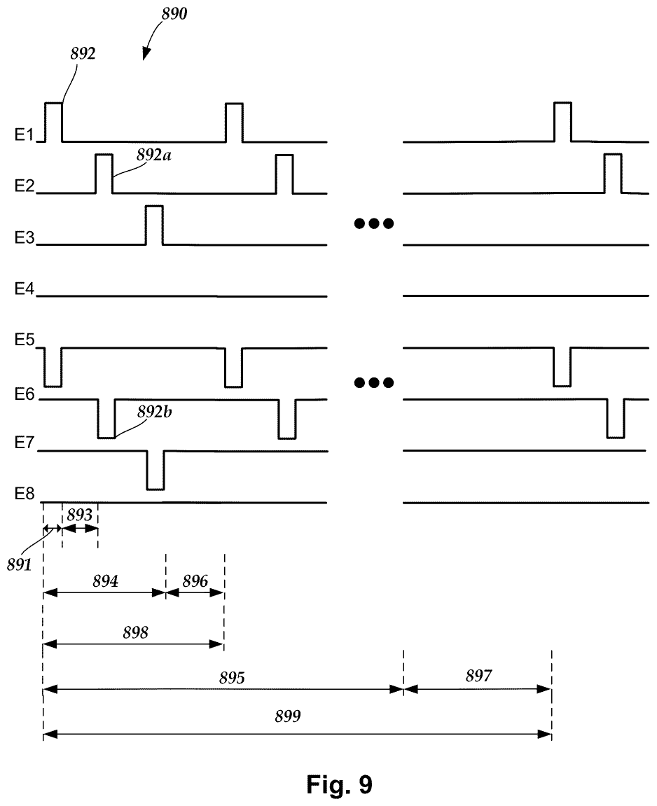

FIG. 9 illustrates a non-limiting example of stimulation using a cuff lead, although it will be understood that similar stimulation can be delivered using any other type of lead. This stimulation follows a microbursting scheme that can be used, at least in some embodiments, for providing targeted therapeutic stimulation or modulation. Further information about, and examples of, the microbursting scheme can be found in U.S. Provisional Patent Application Ser. No. 63/691,918, which is incorporated herein by reference in its entirety.

In at least some embodiments, the microbursting scheme can be used for identifying stimulation targets relative to the electrodes of the cuff or for mapping a nerve. In FIG. 9, a temporal pattern 890 of stimulation pulses 892 is presented for each of eight electrodes E1 to E8 (see, e.g., cuff 450 of FIG. 5). The temporal pattern 890 includes microbursts 894 of stimulation pulses 892.

The microbursting scheme includes a series of temporally-separated pulses 892 generated by the stimulation system and delivered through the electrodes of the cuff. A pulse can be applied to any number of electrodes including, but not limited to, one, two (see, FIG. 9), three, or more electrodes. In at least some embodiments, at a given time, a pulse of the same polarity can be applied to two or more electrodes. In at least some embodiments, at a given time, a pulse of one polarity (e.g., positive pulse 892a) can be applied to one, two, three, or more electrodes and a pulse of another polarity (e.g., negative pulse 892b) can be applied to one, two, three or more other electrodes.

When a pulse is applied to multiple electrodes, the total amplitude of the pulse is distributed among the electrodes. The distribution can be described as “fractionalization”. In at least some embodiments, when a pulse is applied to multiple electrodes, the amplitude of the fractionalization of each electrode can be the same for some or all of the electrodes or can be different between two or more of the electrodes.

In at least some embodiments, at any time, the stimulation provided by the cuff lead can be monopolar, bipolar, or multipolar (e.g., when three or more electrode are activated as a combination of alternating anodes/cathodes). In at least some embodiments, the stimulation can be anodic, cathodic, or a combination thereof. In at least some embodiments, the case of the IPG 14 (FIG. 1) or an external electrode that is relatively distant from the electrodes of the cuff 150 (FIG. 2) can act as an electrode (e.g., a return electrode). Although square pulses are illustrated in FIG. 9, it will be understood that any other suitable waveform can be used, such as for example, sinusoidal or triangular waveforms.

A microburst 894 includes a sequence of temporally-separated pulses 892. A microburst 894 can have any suitable number of temporally-separated pulses 892 including, but not limited to, two, three, four, five, six, seven, eight, ten, twelve, or sixteen or more pulses. In FIG. 9, the microburst 894 includes the following sequence of three bipolar pairs of stimulation pulses: E1(positive)/E5(negative); E2(positive)/E6(negative); E3(positive)/E7(negative).

In at least some embodiments, when stimulation is targeted, a lower stimulation amplitude, as compared to the amplitude typically used for conventional nerve stimulation, can be used, particular when one or more electrodes proximal to the nerve target are selected. In at least some embodiments, each pulse 892 of a microburst 894 has an amplitude of at least 5, 10, or 20 μA. In at least some embodiments, each pulse 892 of a microburst 894 has an amplitude no greater than 1000, 500, 250, 100, 50, or 30 μA. In at least some embodiments, each pulse 892 of a microburst 894 has an amplitude in a range of 5 to 100 μA, 5 to 50 μA, 10 to 50 μA, or 10 to 30 μA. When a pulse is applied to multiple electrodes, this amplitude is distributed among the electrodes. In at least some embodiments, the pulses 892 of a microburst 894 have the same amplitude. In other embodiments, at least two of the pulses 892 of a microburst 894 have different amplitudes.

In at least some embodiments, pulses of different frequency can stimulate different types of axons, fibers, fascicles, or tracts. In at least some embodiments, changing the timing of the pulses or pulse patterns can facilitate specificity in recruiting, stimulating, or modulating specific axons, fibers, fascicles, or tracts. Targeting specific axons, fibers, fascicles, or tracts may also reduce or prevent overstimulation of an area of the nerve. In at least some embodiments, each pulse 892 has a pulse width 891 (i.e., duration) of at least 10, 20, or 30 μs. In at least some embodiments, each pulse 892 has a pulse width 891 (i.e., duration) of no more than 1 ms, 500 μs, 100 μs, 50 μs, or 30 μs. In at least some embodiments, each pulse 892 of a microburst 894 has a pulse width 891 (i.e., duration) in a range of 5 to 100 μs, 5 to 50 μs, or 10 to 30 μs. In at least some embodiments, the pulses 892 of a microburst 894 have the same pulse width. In other embodiments, at least two of the pulses 892 of a microburst 894 have different pulse widths.

In at least some embodiments, a separation period 893 between consecutive pulses 892 of a microburst 894 is at least 10, 20, 30, or 50 μs. In at least some embodiments, the separation period 893 between consecutive pulses 892 of a microburst 894 is no more than 500, 250, 100, or 50 μs. In at least some embodiments, the separation periods 893 between consecutive pulses 892 of a microburst 894 are the same. In at least some embodiments, the separation periods 893 between two pairs of consecutive pulses 892 of a microburst 894 are different. In at least some other embodiments, two or more pulses 892 of a microburst 894 can partially overlap in time.

In at least some embodiments, each microburst 894 is followed by a microburst delay 896 (e.g., an intraburst delay). In at least some embodiments, the microburst delay is at least 10, 20, 30, 50, or 100 μs. In at least some embodiments, the microburst delay is no more than 2000, 1000, 500, 250, or 100 μs. In at least some embodiments, the microburst delay 896 is larger than the separation period 893 between consecutive pulses 892 of the microburst 894. In at least some embodiments, the microburst delay 896 is two, three, or four times larger than the separation period 893 between consecutive pulses of the microburst 894. In at least some embodiments, the microburst delay 896 for each of the microbursts 894 is the same. In other embodiments, the microburst delays 896 for at least two microbursts 894 are different.

The combination of the microburst 894 and microburst delay 896 defines the microburst duration 898. In at least some embodiments, the microburst duration 898 is at least 0.1, 0.5, 1, 5, 10, 20, 30, 50, 70, 90, 100, or 120 seconds. In at least some embodiments, the microburst duration 898 of each of the microbursts 894 is the same. In other embodiments, the microburst durations 898 of at least two microbursts 894 are different.

A macroburst 895 includes multiple microbursts 894, corresponding microburst delays 896, and a macroburst delay 897. The combination of the microburst durations 898 of the corresponding microbursts 894 and the macroburst delay 897 defines the macroburst duration 899. In at least some embodiments, a macroburst 895 can be repeated or a different microburst can be initiated. In at least some embodiments, each macroburst 895 has an identical sequence of microbursts 894 and stimulation pulses 892. In at least some embodiments, at least two consecutive macrobursts 895 have an identical sequence of microbursts 894 and stimulation pulses 892. In at least some embodiments, at least two consecutive macrobursts 895 have nonidentical sequences of microbursts 894 or stimulation pulses 892. In at least some embodiments, at least two consecutive macrobursts 895 have the same microbursts 894 or stimulation pulses 892 (i.e., identical in electrode selection and amplitude) in different temporal order.

In at least some embodiments, the macroburst duration 899, macroburst delay 897, or both are selected to reduce detrimental effects or side effects of continuous microburst stimulation. For example, in some instances, prolonged stimulation of a nerve may result in habituation of the nerve or result in the nerve no longer responding to the stimulation. As another example, in some instances, prolonged stimulation of the vagus nerve can result in esophageal constriction which can produce coughing or vomiting.

In at least some embodiments, the macroburst delay 897 is at least 10, 20, 30, 50, 60, 70, 90, 100, 120, 150, 180, 200, 210, or 240 seconds. In at least some embodiments, the macroburst delay 897 is no more than 1, 2, 3, 4, 5, 6, 7, 8, 9, 10, 12, 15, 20, or 30 minutes. In at least some embodiments, the macroburst delay 897 for each of the macrobursts 895 is the same. In other embodiments, the macroburst delays 897 for at least two macrobursts 895 are different.

In at least some embodiments, the macroburst duration 899 is at least 15, 20, 30, 50, 60, 70, 90, 100, 120, 150, 180, 200, 210, or 240 seconds. In at least some embodiments, the macroburst duration 899 is no more than 1, 2, 3, 4, 5, 6, 7, 8, 9, 10, 12, 15, 20, or 30 minutes. In at least some embodiments, the macroburst duration 899 is in a range of 15 seconds to 10 minutes or in a range of 30 seconds to 8 minutes or in a range of 1 to 5 minutes. In at least some embodiments, the macroburst duration 899 of each of the macrobursts 895 is the same. In other embodiments, the macroburst durations 899 of at least two macrobursts 895 are different.

In at least some embodiments, each microburst 894 of a macroburst 895 has an identical sequence of stimulation pulses 892. In at least some embodiments, at least two consecutive microbursts 894 of a macroburst 895 have an identical sequence (i.e., identical in electrode selection and amplitude) of stimulation pulses 892. In at least some embodiments, at least two consecutive microbursts 894 of a macroburst 895 have nonidentical sequences of stimulation pulses 892 (i.e., at least one corresponding pulse in each sequence differing in electrode selection or amplitude or any combination thereof). In at least some embodiments, at least two consecutive microbursts 894 of a macroburst 895 have the same pulses 892 (i.e., identical in electrode selection and amplitude) in different temporal order.

As one non-limiting example of parameter selection for the macroburst illustrated in FIG. 9, in at least some embodiments, the amplitude is in a range of 5 to 100 μA, the pulse width is in a range of 10 to 100 μs, the microburst delay is in a range of 10 to 100 μs, the macroburst delay is at least 60 seconds, and the macroburst duration is in a range of 1 to 3 minutes.

In at least some embodiments, a reduction in stimulation amplitude for subsequent pulses 892 can be applied (for example, stepwise) at each microdelay (or set of microdelays) or each macrodelay (or set of macrodelays). In at least some embodiments, such a reduction can reduce the concentration of anodic (or cathodic) current over time. In at least some embodiments, this reduction of the concentration of anodic (or cathodic) current can create an anodic (or cathodic) block or an anodic (or cathodic) break excitation (e.g., an action potential generated by stoppage of hyperpolarizing current). Such a procedure may also facilitate understanding of the impact of stimulation on an organ or brain coupled to the stimulated nerve.

In at least some embodiments, the relationship between one or more electrode(s) 334, 358a, 385b (FIG. 4) and one or more tracts, fibers, or nerve bundles 335 (FIG. 10) or other structures of a nerve or other tissue can be visualized using the electrical signals obtained from electrodes of the electrical stimulation lead to estimate the effect of stimulation on the tissue. Additional visualization may include estimating a stimulation field, charge distribution, center of stimulation, or the like. FIG. 10 illustrates one embodiment of a cuff 350 with electrodes 334 disposed around a nerve 280. An estimated region of stimulation 341 is illustrated. The estimation of the region of stimulation 341 can be made using the electrical signals alone or in combination with calculated stimulation field (e.g., volume of activation (VOA), volume of tissue activated (VTA), stimulation field map (SFM), charge distribution, center of stimulation, or the like) estimations Non-limiting examples of methods for calculating an estimate of a stimulation field (e.g., volume of activation, volume of tissue activated, charge distribution, or the like) can be found at U.S. Pat. Nos. 8,326,433; 8,675,945; 8,831,731; 8,849,632; 8.958,615; and 10,265,528; U.S. Patent Application Publications Nos. 2009/0287272; 2009/0287273; 2012/0314924; 2013/0116744; 2014/0122379; and 2015/0066111, all of which are incorporated herein by reference in their entireties. Additional examples of visualization methods can be found in U.S. Provisional Patent Application Ser. No. 63/672,566, incorporated herein by reference in its entirety.

In at least some embodiments, the indication of the relationship between a particular electrode 334, 358a, 358b (or group of electrodes) and each of one or more tracts, fibers, or nerve bundles 335 or body structures can also indicate one or more characteristics of the relationship including, but not limited to, the relative strength of the relationship, whether the electrode activation is beneficial (e.g., a therapeutic effect) or detrimental (e.g., a side effect); whether the electrode activation produces a neural activation effect or a neural blocking effect; or the like or any combination thereof. In at least some embodiments, one or more of the characteristics can also depend on one or more other stimulation parameters, such as, for example, amplitude (total or specific to an electrode), pulse width, pulse frequency, duration of electrode activation, or the like or any combination thereof. Any suitable method for indicating the relationship can be used including, but not limited to, different colors, different brightness, different graphical shapes or other graphical indicia, or the like or any combination thereof. In at least some embodiments, one or more of these characteristics may depend upon a selection of one or more stimulation parameters, such as, for example, stimulation amplitude (total or for individual electrodes), pulse width, stimulation duration, or the like or any combination thereof.

In at least some embodiments, a method, device, or system, as described herein, can be used for mapping a nerve to facilitate identification of target axons, fascicles, nerve fibers, other nerve regions, or tracts for stimulation and which one or more electrodes can be used to stimulate the target. In at least some embodiments, the electrodes can be identified or grouped based on the axons, fascicles, nerve fibers, other nerve regions, or tracts that can be stimulated using the electrode or group of electrodes or peripheral organs or other regions of the body that can be affected by stimulation of the nerve. In at least some embodiments, the mapping can identify or estimate the position, proximity, or directionality (e.g., the direction to the nerve component of interest from the electrode(s)) of one or more of the axons, fascicles, nerve fibers, other nerve regions, or tracts relative to one or more of the electrodes. In at least some embodiments, the mapping can facilitate identifying one or more electrodes for stimulation of one or more target axons, fascicles, nerve fibers, other nerve regions, or tracts or for producing therapeutic effects in one or more peripheral organs of other regions of the body. In at least some embodiments, side effects arising from stimulation by one or more of the electrodes can be identified and inform the selection of one or more electrodes to stimulate one or more target axons, fascicles, nerve fibers, other nerve regions, or tracts or produce therapeutic effects in one or more peripheral organs of other regions of the body.

In at least some embodiments, a method, device, or system can include, or allow a user to define, one or more particular microburst or macroburst arrangements for mapping the nerve or portions of the nerve, for example, to identify one or more electrodes for stimulation of one or more target axons, fascicles, nerve fibers, other nerve regions, or tracts or for production of therapeutic effects in one or more peripheral organs of other regions of the body. In at least some embodiments, a method, device, or system can include incorporation of medical imaging (e.g., computed tomography (CT) imaging or magnetic resonance imaging (MRI)) or medical images to assist, facilitate, verify, or enhance the mapping. Non-limiting examples of mapping methods, systems, and paradigms can be found in U.S. Provisional Patent Application Ser. No. 63/691,918, which is incorporated herein by reference in its entirety.

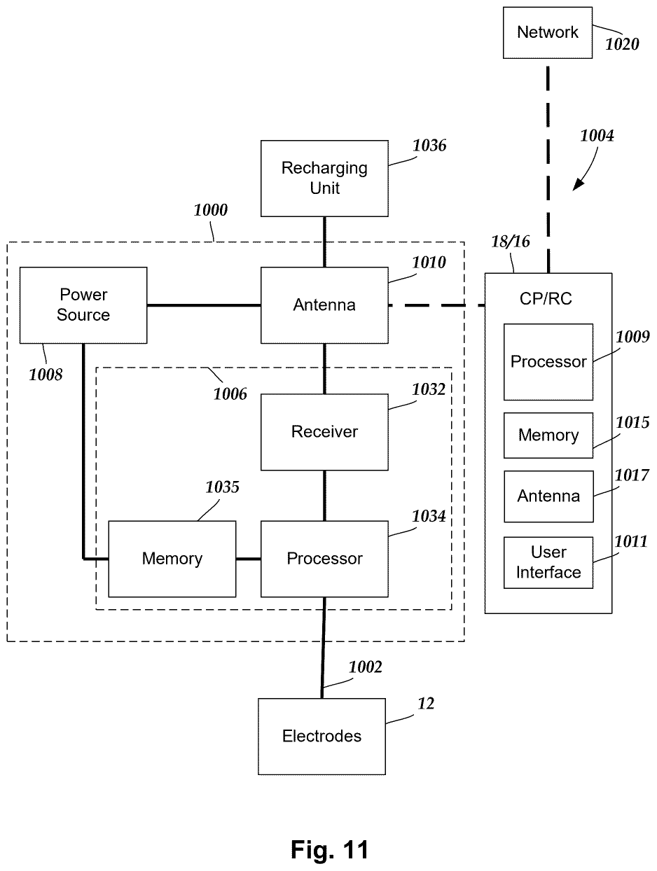

FIG. 11 is a schematic overview of one embodiment of components of an electrical stimulation system 1004 that includes a lead 1002 and a control module 1000 (such as IPG 14 or ETS 20 of FIG. 1) with stimulation circuitry 1006, a power source 1008, and an antenna 1010. The electrical stimulation system can be, for example, any of the electrical stimulation systems described above. It will be understood that the electrical stimulation system can include more, fewer, or different components and can have a variety of different configurations including those configurations disclosed in the stimulator references cited herein.

An external device, such as a clinician programmer (CP) or remote control (RC) 18/16 (or any other suitable device or devices), can include a processor 1009, memory 1015, an antenna 1017, and a user interface 1011. The user interface 1011 can include, but is not limited to, a display screen on which a digital user interface can be displayed and any suitable user input device, such as a keyboard, touchscreen, mouse, track ball, or the like or any combination thereof.

Any power source 1008 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,1123, incorporated herein by reference in its entirety.

If the power source 1008 is a rechargeable battery or chargeable capacitor, the power source may be recharged/charged using the antenna 1010, if desired. Power can be provided for recharging/charging by inductively coupling the power source 1008 through the antenna 1010 to a recharging unit 1036 external to the user. Examples of such arrangements can be found in the references identified above.

In at least some embodiments, electrical current is emitted by the electrodes (such as electrodes 12 in FIG. 1) on the lead 1002 to stimulate nerve fibers, muscle fibers, or other body tissues near the electrical stimulation system. The stimulation circuitry 1006 can include, among other components, a processor 1034, a memory 1035, and a receiver 1032. The processor 1034 is generally included to control the timing and electrical characteristics of the electrical stimulation system. For example, the processor 1034 can, if desired, control one or more of the timing, frequency, strength, duration, and waveform of the pulses. In addition, the processor 1034 can select which electrodes can be used to provide stimulation, if desired. In some embodiments, the processor 1034 selects which electrode(s) are cathodes and which electrode(s) are anodes.

Any processor 1034 can be used and can be as simple as an electronic device that, for example, produces pulses at a regular interval or the processor can be capable of receiving and interpreting instructions from the CP/RC 18/16 that, for example, allows modification of pulse characteristics. In the illustrated embodiment, the processor 1034 is coupled to a receiver 1032 which, in turn, is coupled to the antenna 1010. This allows the processor 1034 to receive instructions from an external source to, for example, direct the pulse characteristics and the selection of electrodes, if desired. Any suitable processor 1009 can be used for the CP/RC 18/16.

Any suitable memory 1035, 1015 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In at least some embodiments, the antenna 1010 is capable of receiving signals (e.g., RF signals) from an antenna 1017 of a CP/RC 18/16, which is programmed or otherwise operated by a user. The CP/RC 18/16 can be a device that is worn on the skin of the user or can be carried by the user and can have a form similar to a pager, cellular phone, or remote control, if desired. As another alternative, the CP/RC 18/16 may not be worn or carried by the user but may only be available at a home station or at a clinician's office.

The signals sent to the processor 1034 via the antenna 1010 and the receiver 1032 can be used to modify or otherwise direct the operation of the control module 1000. For example, the signals may be used to modify the pulses of the electrical stimulation system such as modifying one or more of pulse duration, pulse frequency, pulse waveform, and pulse strength. The signals may also direct the control module 1000 to cease operation, to start operation, t to start signal acquisition, to stop signal acquisition, to start charging the battery, or to stop charging the battery.

Optionally, the control module 1000 may include a transmitter (not shown) coupled to the processor 1034 and the antenna 1010 for transmitting signals back to the CP/RC 18/16 or another unit capable of receiving the signals. For example, the control module 1000 may transmit signals indicating whether the control module 1000 is operating properly or not or indicating when the battery needs to be charged or the level of charge remaining in the battery. The processor 1034 may also be capable of transmitting information about the pulse characteristics so that a user or clinician can determine or verify the characteristics.

It will be understood that each block of the flowchart illustration, and combinations of blocks in the flowchart illustration and methods disclosed herein, can be implemented by computer program instructions. These program instructions may be provided to a processor to produce a machine or engine, such that the instructions, which execute on the processor, create means for implementing the actions specified in the flowchart block or blocks or engine disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device. The computer program instructions can be stored locally or nonlocally (for example, in the Cloud).

The above specification provides a description of the structure, manufacture, and use of the invention. Since many embodiments of the invention can be made without departing from the spirit and scope of the invention, the invention also resides in the claims hereinafter appended.

What is claimed as new and desired to be protected is:

1. A cable for an electrical stimulation system, the cable comprising:
a connector body defining a connector lumen configured for receiving a proximal end of an implantable electrical stimulation lead or of a lead extension coupled or coupleable to the implantable electrical stimulation lead;
a plurality of connector contacts disposed or disposable along the connector lumen and configured for electrically coupling to terminals of the electrical stimulation lead or lead extension when the proximal end of the electrical stimulation lead or lead extension is received in the connector lumen, wherein the plurality of connector contacts comprises a plurality of first connector contacts and at least one second connector contact, wherein each of the at least one second connector contact is not one of the first connector contacts;
a stimulator cord comprising a first end portion coupled or coupleable to the connector body and a second end portion configured for coupling to a stimulator, the stimulator cord comprising a plurality of stimulator contacts disposed along the second end portion, wherein the stimulation contacts are electrically coupled to the first connector contacts, but not to any of the at least one second connector contact, when the stimulator cord is coupled to the connector body; and
one or more sensor cords, each of the one or more sensor cords comprising a first end portion coupled or coupleable to the connector body and a second end portion configured for coupling to an amplifier or signal acquisition unit, the second end portion of each of the one or more sensor cords comprising a connector electrically coupled to at least one of the at least one second connector contact when the sensor cord is coupled to the connector body.

2. The cable of claim 1, wherein the connector body comprises a plurality of the connector lumens.

3. The cable of claim 1, wherein the cable comprises a plurality of the stimulator cords.

4. The cable of claim 1, wherein the cable comprises a plurality of the sensor cords.

5. The cable of claim 1, wherein each of the at least one second connector contact is electrically isolated from the first connector contacts.

6. The cable of claim 1, wherein at least one of the at least one second connector contact is user-selectable from the connector contacts.

7. An electrical stimulation system, comprising:
the cable of claim 1;
the implantable electrical stimulation lead configured to electrically couple to the cable and comprising a plurality of electrodes; and
the stimulator configured to electrically couple to the stimulator cord of the cable.

8. The electrical stimulation system of claim 7, wherein the implantable stimulation lead comprises an implantable cuff lead.

9. The electrical stimulation system of claim 7, wherein the stimulator is an external stimulator.

10. The electrical stimulation system of claim 7, further comprising the amplifier configured to electrically couple to the one or more sensor cords of the cable.

11. The electrical stimulation system of claim 7, further comprising the signal acquisition unit configured to electrically couple to the one or more sensor cords of the cable.

12. The electrical stimulation system of claim 11, wherein the electrical stimulation system is configured to sense electrical signals from tissue using one or more of the electrodes of the electrical stimulation system and deliver the electrical signals to the signal acquisition unit via the cable and the one or more sensor cords of the cable.

13. The electrical stimulation system of claim 12, wherein the electrical stimulation system is configured to analyze the sensed electrical signals.

14. The electrical stimulation system of claim 13, wherein the electrical stimulation system is configured to alter one or more stimulation parameters in response to the analysis of the sensed electrical signals.

15. The electrical stimulation system of claim 7, wherein the at least one second connector contact is electrically isolated from the stimulator when the stimulator is electrically coupled to the cable.

16. A method for stimulating tissue of a patient using the electrical stimulation system of claim 7, the method comprising:
electrically coupling the electrical stimulation lead to the cable;
electrically coupling the cable to the stimulator;
generating, by the stimulator, stimulation signals; and
delivering the stimulation signals to the tissue through the stimulator cord of the cable to one or more of the electrodes of the electrical stimulation lead for stimulation of the tissue adjacent the one or more of the electrodes of the electrical stimulation lead.

17. The method of claim 16, further comprising, after the delivering, sensing electrical signals from the tissue using one or more of the electrodes of the electrical stimulation lead and delivering the electrical signals through the at least one sensor cord of the cable to an amplifier.

18. The method of claim 16, further comprising, after the delivering, sensing electrical signals from the tissue using one or more of the electrodes of the electrical stimulation lead and delivering the electrical signals through the at least one sensor cord of the cable to a signal acquisition unit.

19. The method of claim 18, further comprising analyzing the delivered electrical signals.

20. The method of claim 19, further comprising altering one or more stimulation parameters in response to the analysis of the delivered electrical signals.
